(19) United States
(12) Patent Application Publication
(10) Pub. No.: US 2024/0027441 A1
(43) Pub. Date: Jan. 25, 2024

(54) DEVICES AND METHODS FOR PERFORMING LATERAL FLOW TESTS

(71) Applicant: éclateral Limited, Bristol (GB)

(72) Inventors: Benjamin James Edwards, Bristol (GB); Despina Moschou, Bristol (GB); Paul Ko Ferrigno, Bristol (GB); Pedro Estrela, Bristol (GB); Sarah May Olivia Chapman, Bristol (GB); Uro{hacek over (s)} Zupancic, Bristol (GB)

(21) Appl. No.: 18/548,745

(22) PCT Filed: Mar. 2, 2022
(86) PCT No.: PCT/GB2022/050558

§ 371 Date: Sep. 1, 2023

(30) Foreign Application Priority Data
Mar. 3, 2021 (GB) 2102978.0

(51) Int. Cl. G01N 33/543

(52) U.S. Cl. CPC G01N 33/54388; G01N 33/5438

(57) ABSTRACT

The present disclosure relates to a lateral flow test device for performing a lateral flow test on a liquid sample, comprising: a test strip which comprises: a nitrocellulose membrane having a first capturing reagent disposed on a first surface along a test line, the first capturing reagent being configured to capture a first analyte in the liquid sample; a sample pad disposed at a first end of the nitrocellulose membrane configured to receive the liquid sample; a labelling reagent comprising a plurality of label molecules disposed on the first surface at a position between the sample pad and the test line, the label molecules being configured to bind to the first analyte; and an electrode array disposed over the nitrocellulose membrane configured to apply an electric potential across the first surface.

### FIELD OF THE INVENTION

The present disclosure relates to lateral flow tests.

### BACKGROUND

Lateral Flow Tests (LFTs), also known as Lateral flow assays (LFAs) and Lateral Flow Devices (LFDs), are ubiquitous in the field of medical diagnostics and are widely used in other settings, such as environmental monitoring. They were introduced in the 1980s and are still one of the most cost-effective platforms available for implementation of single use immunosensors (see e.g., Campbell, R. L., Wagner, D. B. and O'Connel, J. P. (1987) Solid-phase assay with visual readout, U.S. Pat. No. 4,703,017; Rosenstein, R. W. and Bloomster, T. G. (1989) Solid-phase assay employing capillary flow, U.S. Pat. No. 4,855,240; cited in O'Farrell (2009) “Evolution in Lateral Flow-Based Immunoassay Systems”, R. C. Wong, H. Y. Tse (eds.), Lateral Flow Immunoassay, 1 DOI 10.1007/978-1-59745-240-3_1). In 2006, more than 200 companies worldwide manufactured LFTs in a market then estimated at $2.1 Bn (O'Farrell, op cit).

An LFT generally comprises a strip made of nitrocellulose that connects a sample pad at one end to a wicking pad at the other end. The configuration ensures an even capillary flow when a liquid sample is applied to the sample pad from the point of deposition, through the nitrocellulose, towards the wicking pad. The configuration is assembled on a backing card for stability. Detection and visualization of the analyte is mediated by a pair of affinity reagents that bind to different surfaces on the analyte. Such pair of affinity reagents may be naturally present as polyclonal antibodies or monoclonal antibodies, or other affinity reagents selected for the purpose. A first reagent of each pair is labelled with an optically detectable reagent such as a microscopic latex bead or a gold nanoparticle and is used in a solution. Typically, the optically detectable label is so small that individual particles are not detectable by eye, but only become detectable by eye when numerous particles accumulate to a very high concentration or amount at a specific location. To achieve this effect, a second (unlabelled) reagent of each pair is immobilized and is used to capture from solution the analyte and any labelled reagent that is bound to the analyte. This capture reagent is not labelled and is printed in a visualization line perpendicular to the direction of flow in what is known as the capture strip. Accumulation of label on this line indicates that the capture reagent has recruited analyte from the solution that has been bound to the labelled reagent.

Typically, two visualisation lines are printed on the nitrocellulose membrane. Closest to the sample pad and the first to be encountered by the sample is the capture reagent, as described above. This is the so-called test line. The second printed line includes an antibody or other capture reagent that directly binds to the labelled reagent and so directly recruits the labelled particle from solution: this is the so-called control line. Since it recruits the labelled component that has been added to the test, the test line should always become visible if the test has been performed correctly. Soluble reagents including the binding affinity reagent labelled with latex beads or gold nanoparticles can be included on the reagent pad during manufacturing, included on a separate reagent pad between the sample pad and the wicking pad, or added to the sample together with a sample buffer prior to application on the membrane.

Although assay performance is tuneable, a number of issues can exist in conventional LFT platforms:
ambiguous interpretation of the result due to subjective visual readout of the sensor's response, leading to moderate clinical performance, and potential exclusion of users with poor eyesight from using the test;
requirement for the amount or concentration of analyte to be above a detectable threshold commensurate with visual detection can sometimes be problematic, for example when the analyte is trace amounts of toxic metals in a sample of drinking water, or if a sample of blood, plasma or serum is required from a patient who is a neonate or elderly person, or a patient with low blood pressure and/or collapsed blood vessels when it is impractical to obtain a large quantity of sample;
limited potential for quantification, meaning that other forms of follow-up tests are often required and that LFTs cannot be used in many situations where a cheap disposable test may be desirable, e.g., in glucose monitoring in diabetic patients;
limited potential for multiplexing, especially when the test is to be used by someone who is not a trained practitioner, meaning that multiple LFTs must be used when more than one analyte or biomarker needs to be detected in a test.
miniaturization of sample volume requirements below microliter level has not been achieved;
integration with onboard electronics and built-in QC functions can be challenging;
test-to-test reproducibility can be challenging.

In view of the foregoing, the present technology provides improved devices and methods for performing lateral flow tests.

### SUMMARY OF THE INVENTION

The present technology thus provides a device and a system of for performing electrochemical lateral flow tests that are non-optical-based and can be implemented on off-the-shelf LFTs. Moreover, the present technology enables the manufacturing of new LFTs in cases where the introduction of LFTs has not been practical, for example, when testing requires high sensitivity, quantification, or multiplexing. The present technology further provides a device and a method for producing and capturing electrochemical signal from the results of conventional LFTs. The captured data may, for example, be transmitted for analysis, storage or onward transmission, e.g. to a computer, a display, a smartphone, other smart devices, data storage, or a transmission device, etc. The present technology further provides a computer-implemented method of interpreting LFT results for a user.

An aspect of the present technology provides a lateral flow test device for performing a lateral flow test on a liquid sample, comprising: a test strip which comprises: a nitrocellulose membrane having a first capturing reagent disposed on a first surface along a test line, the first capturing reagent being configured to capture a first analyte in the liquid sample; a sample pad disposed at a first end of the nitrocellulose membrane configured to receive the liquid sample; a labelling reagent comprising a plurality of label molecules disposed on the first surface at a position between the sample pad and the test line, the label molecules being configured to bind to the first analyte; an electrode array disposed over the nitrocellulose membrane configured to apply an electric potential across the first surface; and a housing enclosing the test strip and the electrode array, the housing comprises a support element configured to support the electrode array and the test strip in an initial position wherein the electrode array is spaced apart from the test strip.

In some embodiments, the housing may comprise a compressible portion configured to displace the electrode array from the initial position when the compressible portion is compressed.

In some embodiments, the support element may comprise a pivot coupled to the electrode array to allow the electrode array to rotate.

In some embodiments, the support element may further comprise a pin coupled to a rear end of the electrode array, the pin extending through a linear cut-out in the housing, wherein movement of the pin rotates the electrode array about the pivot.

In some embodiments, the housing may further comprise a wedge element, wherein, upon compression, the wedge element compresses the compressible portion to rotate the electrode array about the pivot.

In some embodiments, the electrode array may comprise a core formed of an insulating material and a plurality of electrodes disposed on the core.

In some embodiments, the core and/or the electrode array may be formed of a flexible material.

In some embodiments, the electrode array may have an opening to allow the electrode array to flex.

In some embodiments, the housing may comprise a tilted section configured to support the test strip in a tilted position.

In some embodiments, the device may further comprise a wicking pad having a high liquid absorption capacity disposed at a second end of the nitrocellulose membrane and configured to provide a capillary force to drive the liquid sample from the sample pad along the nitrocellulose membrane.

In some embodiments, the device may further comprise a conjugate pad disposed on the first surface of the nitrocellulose membrane impregnated with the labelling reagent.

In some embodiments, the nitrocellulose membrane may further comprise a second capture reagent disposed on the first surface along a control line, the second capture reagent being configured to capture the label molecules.

In some embodiments, the plurality of electrodes may comprise one or more working electrodes arranged to overlay the test line and a background portion of the nitrocellulose membrane, the one or more working electrodes being configured to apply an electric potential on the first surface of the nitrocellulose membrane.

In some embodiments, the plurality of electrodes may comprise one or more counter electrodes each arranged to oppose a corresponding working electrode to complete an electric circuit.

In some embodiments, the plurality of electrodes may comprise one or more reference electrodes configured to act as a point of reference for the potential applied by the working electrodes.

In some embodiments, the or each reference electrode may be disposed adjacent a counter electrode.

In some embodiments, at least one working-electrode-counter-electrode pair may be arranged in a depression of the electrode array such that when in contact with the test strip, the depression forms a sampling well.

In some embodiments, the electrode array may be disposed on a printed circuit board, PCB.

In some embodiments, the PCB may comprise a plurality of conductive layers.

In some embodiments, one or more conductive layers of the plurality may be covered with a solder mask layer.

In some embodiments, the label molecules may comprise molecules that exhibit a catalytic activity such as gold nanoparticles.

In some embodiments, the device may further comprise a signal enhancer disposed on the first surface of the nitrocellulose membrane or the sample pad configured to precipitate on a surface of the label molecules for electrochemical detection of the label molecules.

In some embodiments, the device may further comprise an additional conjugate pad disposed on the first surface of the nitrocellulose membrane impregnated with the signal enhancer.

In some embodiments, the signal enhancer may comprise a plurality of metallic ions, optionally the plurality of metallic ions is derived from a silver salt.

In some embodiments, the electrode array may comprise a plurality of gold-plated electrodes, and wherein the plurality of gold-plated electrodes disposed over the nitrocellulose membrane initiates a reduction of silver ions derived from the silver salt on the gold-plated electrodes.

In some embodiments, the plurality of metallic ions may be provided in combination with a reducing agent.

In some embodiments, the reducing agent may be a hydroquinone solution.

In some embodiments, the device may be provided with a unique identifier, the unique identifier optionally comprises a barcode, a QR code, or a combination thereof.

In some embodiments, the housing may be provided with a window above the test line for visual evaluation of the test line.

In some embodiments, the device may further comprise a plurality of test strips incorporated within the housing.

In some embodiments, the device may further comprise: an integrated electronic reader configured to generate an electrical signal through the electrode array to drive an electrochemical reaction in the test strip, and to receive a resulting electrochemical signal from the test strip through the electrode array; and a power source configured to power the electrode array through the electronic reader.

In some embodiments, the electrode array may comprise at least one pair of electrodes, wherein the resulting electrochemical signal comprises an electrical current across the at least one pair of electrodes, or a change in conductance, resistance, capacitance or impedance of an electrical circuit between the at least one pair of electrodes, or a combination thereof.

In some embodiments, the device may further comprise a communication interface configured to communicate with an external electronic device to send the received electrochemical signal.

In some embodiments, the first capturing reagent may be further disposed on the first surface the nitrocellulose membrane along a second test line at a concentration different from the first test line.

In some embodiments, the nitrocellulose membrane may further comprise a third capturing reagent disposed on the first surface along a third test line, the third capturing reagent being configured to capture a third analyte in the liquid sample different from the first analyte.

In some embodiments, the electrode array may comprise a plurality of pairs of corresponding electrodes, the plurality of corresponding electrodes being arranged such that a pair of corresponding electrodes of the plurality overlays a background of the nitrocellulose membrane and a pair of corresponding electrodes overlays the first test line, and optionally a pair of corresponding electrodes of the plurality overlays each of the control line and/or the second test line and/or the third test line.

Another aspect of the present technology provides an electronic reader for reading a result from a lateral flow test device, the lateral flow test device comprises a compressible portion, the electronic reader comprising: a receiving portion for receiving the lateral flow test device comprising a releasing mechanism configured to compress the compressible portion of the lateral flow test device; and a communication port configured to electrically couple with the lateral flow test device to generate an electrical signal for driving an electrochemical reaction within the lateral flow test device and to receive a resulting electrochemical signal indicative of the electrochemical reaction.

In some embodiments, the electronic reader may further comprise a power connection for receiving power from an external source.

In some embodiments, the electronic reader may further comprise an integrated power source.

In some embodiments, the electronic reader may further comprise an optical reader for reading a unique identifier on the lateral flow test device.

In some embodiments, the releasing mechanism may comprise a linear actuator configured to compress the compressible portion of the lateral flow test device upon insertion of the lateral flow test device into the receiving portion.

In some embodiments, the electronic reader may further comprise a timer configured to countdown a measurement time defined by a length of time required for measuring the result.

Another aspect of the present technology provides a system for performing a lateral flow test on a liquid sample, comprising: a lateral flow test device comprising: a test strip which comprises: a nitrocellulose membrane having a first capturing reagent disposed on a first surface along a test line, the first capturing reagent being configured to capture a first analyte in the liquid sample; a sample pad disposed at a first end of the nitrocellulose membrane configured to receive the liquid sample; a labelling reagent comprising a plurality of label molecules disposed on the first surface at a position between the sample pad and the test line, the label molecules being configured to bind to the first analyte; an electrode array disposed over the nitrocellulose membrane configured to apply an electric potential across the first surface; and a housing enclosing the test strip and the electrode array, the housing comprises a support element configured to support the electrode array and the test strip in an initial position wherein the electrode array is spaced apart from the test strip; and an electronic reader comprising: a receiving portion for receiving the lateral flow test device comprising a releasing mechanism configured to compress the housing of the lateral flow test device so as to bring the electrode array into contact with the test strip in a read-ready position; and a communication port configured to electrically couple with the electrode array of the lateral flow test device to generate an electrical signal in the electrode array for driving an electrochemical reaction in the test strip, and to measure from the electrode array a resulting electrochemical signal indicative of the electrochemical reaction in the test strip.

In some embodiments, the housing may comprise a compressible portion configured to displace the electrode array from the initial position when the compressible portion is compressed.

In some embodiments, the releasing mechanism may be actuated through action of inserting the lateral flow test device into the receiving portion.

In some embodiments, the support element may comprise a pivot coupled to the electrode array and the releasing mechanism may comprise a protruding element configured to protrude into the compressible portion to rotate the electrode array upon insertion of the lateral flow test device into the receiving portion.

In some embodiments, the protruding element may be biased towards a centre line of the receiving portion by a resilient element, wherein the protruding element may be released into the compressible portion by the resilient element upon insertion of the lateral flow test device into the receiving portion.

In some embodiments, the support element may comprise a pivot coupled to the electrode array to allow the electrode array to rotate and a pin coupled to a rear end of the electrode array, the pin extending through a linear cut-out in the housing; and the releasing mechanism may comprise a guiding groove on an interior surface of the receiving portion, the guiding groove may be angled towards a centre line of the receiving portion and configured to receive the pin to guide the pin towards the centre line upon insertion of the lateral flow test device into the receiving portion to rotate the electrode array.

In some embodiments, the support element may comprise a pivot coupled to the electrode array and the housing further comprises a wedge element, and the releasing mechanism comprises a surface against which the wedge element pushes such that the wedge element compresses the compressible portion to rotate the electrode array upon insertion of the lateral flow test device into the receiving portion.

In some embodiments, the releasing mechanism may comprise a linear actuator configured to extend towards a centre line of the receiving portion upon actuation.

In some embodiments, the releasing mechanism may be configured to compress the compressible portion with a pressure ranging from 1N to 50N.

In some embodiments, the lateral flow test device may comprise a port having an exposed portion of the electrode array, the port being arranged to couple with the communication port of the electronic reader

A further aspect of the present technology provides a method of performing a lateral flow test on a liquid sample using a lateral flow test strip, the lateral flow test strip comprising: a nitrocellulose membrane having a capturing reagent disposed on a first surface along a test line, the capturing reagent being configured to capture a predetermined analyte in the liquid sample; a sample pad disposed at one end of the nitrocellulose membrane configured to receive the liquid sample; and a labelling reagent comprising a plurality of label molecules disposed on the first surface between the sample pad and the test line, the label molecules being configured to bind to the predetermined analyte, the method comprising: applying an electric potential across the first surface through an electrode array disposed over the nitrocellulose membrane to drive an electrochemical reaction in the lateral flow test strip; and measuring a resulting electrochemical signal from the lateral flow test strip through the electrode array.

In some embodiments, the method may further comprise depositing the liquid sample on the sample pad.

In some embodiments, the electrode array may comprise a plurality of pairs of corresponding electrodes, the method may further comprise overlaying a pair of corresponding electrodes over a background of the nitrocellulose membrane and a pair of corresponding electrodes over the test line, and determining a difference in the resulting electrochemical signal between the pair of electrodes overlaying the background and the pair of electrodes overlaying the test line.

In some embodiments, the method may further comprise, before applying an electric potential, washing the nitrocellulose membrane with a buffer through the sample pad.

In some embodiments, the method may further comprise, before applying an electric potential, depositing a signal enhancer solution comprising a plurality of metallic ions to the nitrocellulose membrane through the sample pad.

In some embodiments, the plurality of metallic ions may be derived from a silver salt.

In some embodiments, the method may further comprise an activation step of combining the signal enhancer solution with a developer solution.

In some embodiments, the developer solution may comprise a reducing agent to enable spontaneous reduction of the plurality of metallic ions on the surface of the label molecules.

In some embodiments, the method may further comprise measuring along the first surface of the nitrocellulose membrane a distribution of metal resulting from at least a portion of the plurality of metallic ions precipitated on a surface of the label molecules to determine a concentration of label molecules along the first surface.

In some embodiments, the method may further comprise measuring along the nitrocellulose membrane a distribution of a reagent (e.g. metallic ions) not reacted with (e.g. not precipitated on a surface of) the label molecules.

In some embodiments, the method may further comprise performing the potential sweep for at least two times, wherein a first cycle of potential sweep resets the electrochemical reaction and a second cycle of potential sweep measures the electrochemical signal.

In some embodiments, measuring a resulting electrochemical signal from the lateral flow test strip may comprise performing a potential sweep along the nitrocellulose membrane through the electrode array.

In some embodiments, detection of a current peak may indicate a local concentration of metallic ions.

In some embodiments, detection of an absence of a current peak at or near the test line may indicate presence of the predetermined analyte in the liquid sample.

In some embodiments, measuring a resulting electrochemical signal from the lateral flow test strip may be performed based on a voltametric method, an amperometric method, a potentiometric method, or an impedance-based method, or a combination thereof.

In some embodiments, measuring a resulting electrochemical signal from the lateral flow test strip may be performed using a linear sweep voltammetry method.

In some embodiments, the linear sweep voltammetry method may comprise a potential sweep at a predetermined rate from a minimum potential to a maximum potential with respect to a reference potential, wherein optionally the predetermined rate may be in a range of 10 mV per second to 1000 mV per second.

In some embodiments, the minimum potential with respect to the reference potential may be in a range of −1V to −0.1V, preferably −0.5V.

In some embodiments, the maximum potential with respect to the reference potential may be in a range of +0.1V to +1V, preferably +0.5V.

In some embodiments, the linear sweep voltammetry method may comprise repeating the potential sweep for a predetermined number of times.

In some embodiments, the linear sweep voltammetry method may comprise a deposition step of applying a low potential for a predetermined period of time before performing the potential sweep, and/or applying a high potential for a predetermined period of time after performing the potential sweep.

In some embodiments, the low potential may be in a range of −1V to −0.1V with respect to the reference potential, and/or wherein the high potential is in a range of 0.1V to 1V with respect to the reference potential.

In some embodiments, the predetermined period of time may be less than or equal to one minute.

In some embodiments, measuring a resulting electrochemical signal from the lateral flow test strip may be performed using a pulse based electrochemical technique.

In some embodiments, the pulse based electrochemical technique may comprise a differential pulse voltammetry method or square wave voltammetric method.

### BRIEF DESCRIPTION OF THE DRAWINGS

Embodiments will now be described, with reference to the accompanying drawings, in which:

FIG. 1A shows an exemplary lateral flow assay strip;

FIG. 1B shows an exemplary lateral flow assay with signal amplification using silver enhancement;

FIG. 2 shows an exemplary printed circuit board (PCB) carrying an electrode array with multiple working, counter and reference electrodes;

FIG. 3A shows a bottom side of a PCB carrying an electrode array in a conceptual representation of overlaying an LFT strip with the PCB;

FIG. 3B shows a conceptual representation of overlaying an LFT strip with the PCB of FIG. 3A coming into contact with the LFT strip;

FIG. 4A shows an exploded view of an exemplary cassette encompassing an LFT, PCB electrode array with a top and a bottom cover;

FIG. 4B shows the exemplary cassette of FIG. 4A with exposed connections for electronic readout;

FIG. 4C shows an exemplary electronic reader configured to read the cassette of FIG. 4B;

FIG. 5 shows an exemplary electrode array layout;

FIG. 6 shows an exploded view of an exemplary lateral flow test device;

FIGS. 7A to 7F illustrate exemplary systems of lateral flow test devices and readers according to various embodiments;

FIG. 8A shows separate working electrodes overlaying over the background, the test line and the control line of an LFA during an exemplary signal readout;

FIG. 8B shows the result of a silver reaction on the nitrocellulose membrane of the LFA during the exemplary signal readout of FIG. 5A;

FIG. 8C shows reduction of Ag⁺ on the AuNPs which increases the concentration of metallic silver locally where AuNPs have accumulated due to immunocomplex formation;

FIG. 8D shows the concentration of Ag⁺ being lowered in corresponding areas on the membrane;

FIG. 8E shows, in an example in which gold PCB is placed on the membrane, a potential sweep reveals oxidation peaks of metallic silver deposited on the gold PCB electrodes and the peak is suppressed where AuPNs accumulate;

FIG. 9 shows an exemplary linear sweep voltammetry demonstrating presence of silver oxidation peak in background of a membrane and disappearance of the peak in the test/control line where AuNPs accumulate;

FIG. 10 shows sequential scanning of four PCB electrodes where the first and third electrodes overlay the background, the second overlays the test line, and the fourth overlays the control line, where both test line and control line are positive i.e., have accumulated AuNPs;

FIG. 11 shows a schematic representation of repeating LSV measurements;

FIG. 12 shows an exemplary differential pulse voltammetry;

FIG. 13A shows a representation of visual tests on a commercially available hCG LFT with increasing hCG concentrations

FIG. 13B shows a visual evaluation of brightness in a test/control line of a commercially available hCG LFT in comparison to the background, with brightness along the Y-axis being the ratio of test/control line divided by the background; and

FIG. 13C shows an electrochemical evaluation on the same test strips as FIG. 13B.

### DETAILED DESCRIPTION

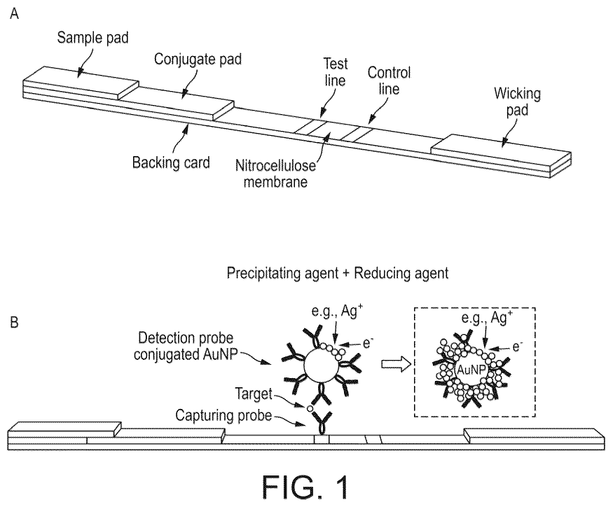

Turning first to FIG. 1A, an exemplary lateral flow test (LFT) comprises a sample pad (where a sample is introduced to the test) overlayed with a conjugate pad. When a sample is introduced, the analyte travels from the sample pad to the conjugate pad due to capillary forces. The conjugate pad is impregnated with a label molecule which could be an antibody/Affimer/aptamer coated gold or latex nanoparticle. Upon introduction, the analyte interacts with a predetermined labelling reagent (e.g. AuNPs gold nanoparticles) and continue to move through the test under capillary forces. The conjugate pad is in contact with a nitrocellulose membrane where a line of capturing reagent (capturing antibody/Affimer/aptamer etc.) is printed. When the analyte of interest flows through the line of capturing reagent (test line), an immunocomplex is formed which attracts the AuNPs to the test line. Another line is printed downstream from the test line (control line) which includes reagents that can capture AuNPs without the presence of the analyte. Immunocomplex formation in the control line act as a confirmation that reagents in the test are uncompromised and the result on the test line is valid. Downstream from the test/control lines, the nitrocellulose membrane connects to a wicking pad, which has a high liquid absorption capacity and drives the capillary actions needed to move the sample and labels through the nitrocellulose membrane.

According to present technologies, sensitivity of the AuNPs-based LFTs may be increased by the exploitation of signal enhancement strategies utilizing enzymatic or non-enzymatic approaches such as silver enhancement, among others. In an embodiment shown in FIG. 1B, the silver enhancement strategy is based on the use of a silver salt (silver lactate/nitrate or acetate) in combination with a reducing agent such as hydroquinone in solution, where gold acts as catalyst enabling electron transfer from the reducing agent to the silver ions, which precipitate on the surface of the gold forming metallic silver. The combined silver salt and reducing agent solution may be provided for example as an additional conjugate pad impregnated with the signal enhancer or as an integrated reservoir within the cassette which can be broken manually or automatically (and the solution within is released). The precipitated silver continues to catalyse the reaction which results in layers of silver being deposited on the gold surfaces, thus enlarging the AuNPs. According to embodiments, increased AuNP size contributes to higher sensitivity of LFTs utilizing silver enhancement by visual evaluation of the test/control line.

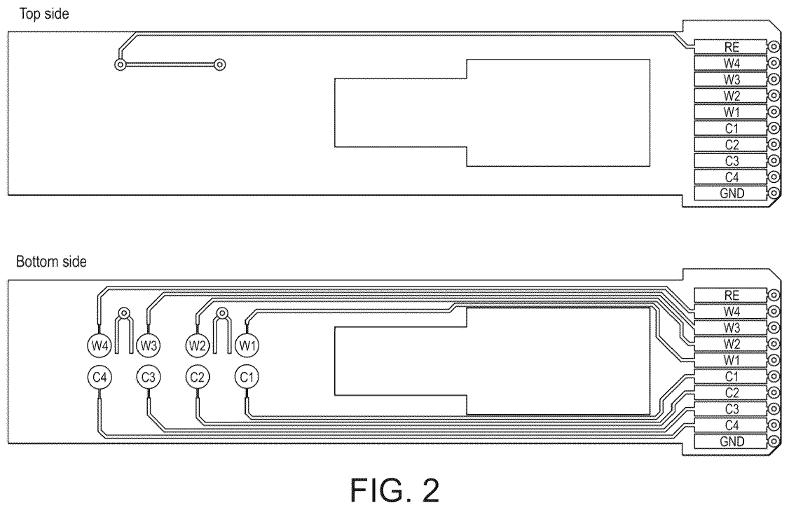

In an embodiment, an electrode array may be constructed and overlayed over the nitrocellulose membrane to capture the signal developed on the test line and the control line in an LFT. The electrode array may be composed of an inert insulating core material and conductive ‘pads’ or electrodes made of conducting metal/polymer/nanomaterial or organic material capable of electrical signal transduction. Additive or subtractive manufacturing techniques may be used for the manufacturing of the electrode array such as, but not limited to photolithography, screen printing, inkjet printing, 3D printing etc. In an embodiment, depicted in FIG. 2, the electrode array may be disposed on a printed circuit board (PCB). The PCB may include multiple conductive layers which may optionally be covered with a thin solder mask layer preventing the exposure of undesired pads. The PCB shown is double-sided carrying conductive connection to the connector part with no interference from the LFT. In an embodiment, the PCB includes multiple electrodes forming a standard three electrode cell configuration, where individual working electrodes (WEs) are overlaid on the test line, control line and background (no line) on the nitrocellulose membrane. Multiple or single counter electrodes (CEs) may also be present on the PCB as well as separate reference electrodes (REs).

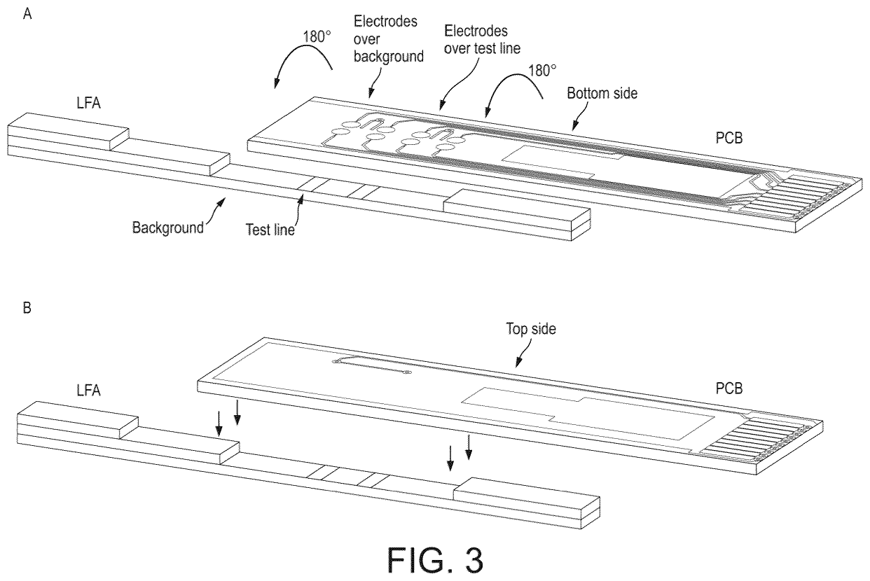

As shown in FIG. 3, the electrode array is placed on top of the LFT to align the strip background, test line and the control line with respective pairs of working and counter electrodes. The bottom side of the PCB comes in contact with the nitrocellulose membrane during the measurement, while other parts of the test (sample pad, conjugate pad, wicking pad) are avoided. In the present embodiment, the electrodes on the electrode array are not functionalized, which enables a long shelf life and allows implementation of the present method with any colloidal gold based LFT since the immunocomplex formation forms within the membrane matrix and not on the surface of the electrodes. In other embodiments, the electrodes on the electrode array may alternatively be functionalized if desired.

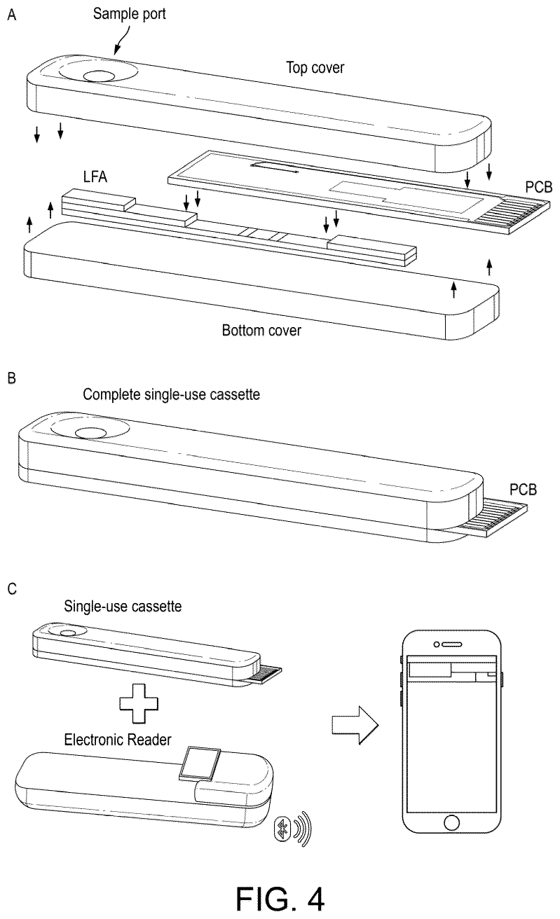

An electronic lateral flow test (eLF) device according to an embodiment is illustrated in FIG. 4A, in which the LFT and the electrode array are sandwiched between a top and a bottom cover forming a housing. The device may e.g. be a single-used cassette and can for example be made of renewable materials such as compostable e.g. corn-based plastics or other natural composites, bamboo, wood and so on. Optionally, the cassette may be provided with a window for visual reading of the test line and the control line, though one is not required since the result is read electronically.

In an embodiment, the cassette has exposed connections arranged to be inserted in an electronic reader for the result to be measured by a reader chip in the electronic reader, as shown in FIG. 4B. In an embodiment, the cassette is provided with a connection component which may include e.g. a unique printed bar code or QR code. The connection component may be arranged to be inserted into a port on the electronic reader to perform the measurement as shown in FIG. 4C. In an alternative embodiment, a reader chip may be incorporated into the cassette together with a communication interface e.g. a Bluetooth chip and a power source such as a Li polymer battery. In either configuration, the integrated electronic LFT (eLF) may be single-use and disposable e.g. for potentially infectious samples, or, in alternative embodiment, the cassette may be opened, the test strip replaced and the cassette reused. Since the electronic reader does not come into contact with the sample, potential contamination is negligible and the reader is therefore reusable.

Once the result is read, the test result may be instantly transmitted from the electronic reader, or from the cassette in embodiments where the electronics have been incorporated within the cassette, either wirelessly e.g. via Bluetooth/WiFi or via physical connections such as a USB port/AUX port-based cables. The test result may be transmitted to e.g. a computer for analysis, to a communication device for onward transmission, to a smartphone or other smart devices with a corresponding app, or a storage device. The data may be stored, analysed and/or transmitted in real time to relevant parties, such as health authorities, health service, carers, employers or in the case of environmental monitoring farmers, utilities, environmental agencies, public health bodies etc.

In an embodiment, the reader may comprise a bespoke chip within a body together with a power source for a portable reader or provided with a power connection for a home-or lab-based reader. The bespoke chip and firmware generate the signal to drive the electrochemical reaction and to capture the resulting electrochemical signal, which may be an electrical current or a change in the properties of an electrical circuit between the working and counter-electrodes, such as its conductance, resistance, capacitance or impedance. Useful signals are obtained when there is a difference between the pair of electrodes aligned with bare membrane (background) and those aligned with the test (expected to be positive if the test is working correctly) and capture lines (which measure the presence of the analyte). It will be clear to those skilled in the art that the characteristics of the electrical circuit will vary in accordance with the number of labelled particles that accumulate at each line, and that the strength of the signal will be a proximate readout of this number and may be used for quantification of the analyte. It is anticipated that multiple electrode pairs may be used to monitor multiple lines on a single strip, and that the use of an electronic reader to perform result measurement such that the result is interpreted digitally means that multiple simultaneous readouts data can be obtained in a single test by an untrained user without the risk of confusion.

In another embodiment, multiple test lines with a range of concentrations of the reagent used to capture the label may be used to calibrate the assay and/or to assist with quantification of the analyte. Moreover, present embodiments enable multiple test strips to be incorporated in a single cassette side-by-side or on top of each other (e.g. separated by PCBs), which is not possible when visual/optical readouts are performed.

The reader may have a single port, so that one test is read at a time, or multiple ports may be provided to allow multiple simultaneous reading for a higher throughput. In one embodiment, the reader incorporates a bar code reader, camera or other optical devices used for reading a unique bar code or QR code or the like printed on the connection component of the disposable or reusable cassette. The reader may incorporate a mechanical button that opens the sample port when the cassette is inserted into the reader. The reader may incorporate a countdown timer to ensure that measurements are taken at appropriate times. This would also enable the use of kinetic measurements that may extend the measurement range of the test, for example allowing an early readout of high levels of analyte that would exceed the maximum limit of the assay if the measurement were allowed to run for the standard amount of time required to measure low levels of analyte in other samples.

An embodiment of the reader is provided that is designed for use in the home. For example, with a single use battery, each reader may run 200 tests, allowing for example a family of 4 to run one test a day each for 50 days. The reader may then be returned to the manufacturer to be reused to increase the sustainability of the reader. In another embodiment, the reader may be connected to an external power source to be used either for a higher number of samples per day, or for an extended duration.

According to present embodiments, an electrode array includes one or more working electrodes (WE), one or more counter electrodes (CE) and one or more reference electrodes (RE). The working electrodes are arranged to align over the test line and the control lines on the nitrocellulose membrane, while a counter electrode is provided to complete the circuit with a corresponding working electrode. According to present embodiments, the WEs are smaller than the test and control lines on the nitrocellulose membrane to ensure proper alignment.

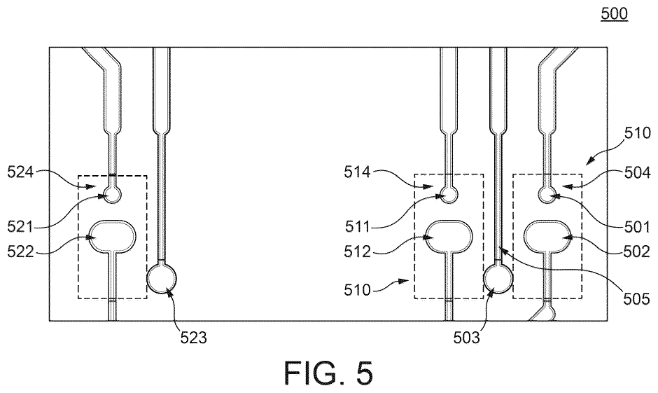

FIG. 5 shows an exemplary electrode array 500 according to an embodiment. The electrode array 500 comprises a plurality of working electrodes 501, 511 and 521, a plurality of counter electrodes 502, 512, 522, and a plurality of reference electrodes 503, 523, disposed on a dielectric core 510.

In the present embodiment, WE 501 is arranged to measure the reagent in the background of the nitrocellulose membrane. CE 502 is disposed opposite to and in close proximity with WE 501; however, other configurations are possible which will be apparent to a person skilled in the art. RE 503 is disposed adjacent WE 501 and CE 502 to ensure a stable application of potential on WE 501. The three electrodes WE 501, CE 502 and RE 503 form the components of a sampling well 504 (shown in dotted line). The sampling well 504 is lower in height compared to the dielectric core 510 to enable fluid sample to accumulate within the sampling well 504 and around the electrodes WE 501 and CE 502. The difference in height may be achieved using any suitable and desirable techniques on dielectric material such as solder mask, any photoresist, or injection moulding.

WE 511 and CE 512 form the components of a second sampling well 514 with RE 503. The two sampling wells 504 and 514 share the same RE 503. In the present embodiment, the majority of reference electrode trace 505 is covered by dielectric 510 to reduce the likelihood of a reaction occurring on the electrode surface of the RE 503, which can impact the reaction at WE 501 and WE 511 when an RE is in close proximity with a WE. For the same reason, RE 503 is disposed on the side of CE 502 and CE 512 to reduce the likelihood of the reactions occurring on RE 503 interfering with the reactions occurring on WE 501 and WE 511.

A third WE 521 and corresponding CE 522 are disposed within a third sampling well 524, provided with a second RE 523.

In the present embodiment, CE 502, CE 512 and CE 522 may be connected to the same output and RE 503 and RE 523 may also be connected to the same pin in the analogue front end (AFE).

According to present embodiments, various mechanisms may be employed to bring the electrode array into contact with the nitrocellulose membrane to create isolated sample wells to determine a concentration of a reagent. It is noted that the concentration of the reagent is proportional to a concentration of an analyte due to local immunocomplexes formed on the nitrocellulose membrane. In some embodiments, compression may be applied to the lateral flow test device to improve performance at the sampling wells, which will be discussed below.

In some embodiments, a 0.8 mm FR-4 core can be used as a suitable dielectric core, which can be populated with copper traces and pads and undergo ENIG plating. Alternative plating techniques may include electrolytic or electroless plating, which can be applied with materials ranging from gold, silver, platinum etc. In general, higher thickness of plating increases the reliability of the sensors. The plating thicknesses are conventionally described in microinches, where 1, 2, and 3 microinch plating are of particular interest. The varying surface finish will be apparent to a person skilled in the art.

In present embodiments, the dielectric material is a fiberglass-based core which provides a degree of flexibility. Alternatively or in addition, a flexible electrode array may be used based on a polyamide film known as flex PCB. Screen spiting or inkjet printing, amongst other techniques, may also be used to deposit conducting material on a rigid or flexible dielectric to form part of the lateral flow test device assembly or directly printed on the housing of the lateral flow test device assembly base (top or bottom). The electrode array within the LFT device enables electrical connection with an electronic reader without the electronic reader coming into contact with the sample within the LFT device, thus enabling the electronic reader to be safely reused.

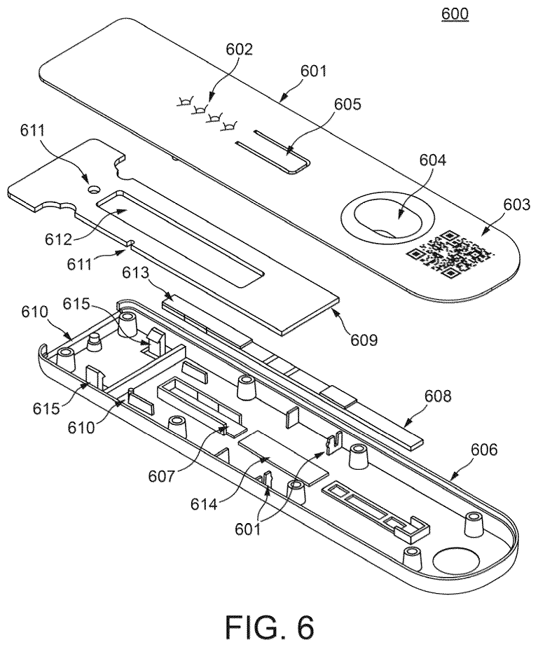

In embodiments of the present technology, the electrode array of an LFT device is supported in its initial position that is spaced apart from the nitrocellulose membrane of the LFT device, such that, initially, the electrode array is not in contact with the nitrocellulose membrane. After a liquid sample has been deposited in the sample port of the LFT device, the electrode array is brought into contact with the nitrocellulose in a read-ready position. FIG. 6 shows the construction of an exemplary lateral flow test device 600 in an exploded view according to an embodiment.

The device 600 comprises a housing including a top portion 601 and bottom portion 606. The top portion 601 of the housing is provided with ventilation holes 602, QR code 603 that uniquely identifies the device 600 to allow the test to be linked to a particular person, a sample port 604 for receiving a liquid sample, and a compressible portion 605. The top portion 601 is configured to mechanically couple to the bottom portion 606, which forms a bed 607 for the lateral flow array strip (nitrocellulose membrane) 608 to ensure proper alignment of the LFA strip 608.

An electrode array 609 overlays the LFA strip 608 and is provided with alignment features 611, which sits within opposing alignment features 610 of the bottom portion 606 to ensure minimal movement of the electrode array in the horizontal direction (parallel to the plane of the bottom portion 606). The electrode array 609 is provided with an opening 612 to allow an absorption pad 613 of the LFA strip 608 to expand as liquid is absorbed. The opening 612 further enables the electrode array 609 to flex. When the compressible portion 605 is compressed, it displaces the electrode array 609 from the initial position towards the LFA strip 608 and brings the electrode array 609 into contact with the LFA strip 608 is the read-ready position. The flexibility provided by the opening 612 of the electrode array 609 facilitates such a displacement.

The transition from the initial position to the read-ready position results in a non-horizontal positioning of the electrode array 609. To ensure uniform contact between the electrode array 609 and the LFA strip 608, the bed 607 is configured with a tilted section 614 to counter the non-horizontal positioning of the electrode array 609 in the read-ready position. Support features/elements 615 are provided to the bottom portion 606 configured to support the electrode array 609 in the initial position spaced apart from the LFA strip 608, and to hold the electrode array 609 in a correct position when it is brought into contact with the LFA strip 608 by the compressible portion 605, thus allowing the transition of the electrode array 609 between the two positions with control.

An important consideration is the pressure exerts on the LFA strip 608 when it comes into contact with the electrode array 609 through mechanical compression of the compressible portion 605. The Applicant has recognized that varying the pressure has varying effects on the electrochemical reading of the LFA strip 608. When the LFA strip 608 is compressed with a low pressure (e.g. 1N), electrical contact between the LFA strip 608 and the electrode array 609 is made, and measurements can be taken. When a higher pressure is exerted (e.g. 5N) the electrochemical measurements become more reproducible due to a more uniform pressure distribution along the LFA strip 608 and electrode array 609. If an even higher pressure is used (e.g. 30N), the membrane becomes more compressed, and the flow of sample liquid through the nitrocellulose membrane during a measurement is more effectively stopped, such that isolation of localised sampling pools created by the indentations/depressions of the electrode array 609 becomes easier leading to higher sampling resolution. Moreover, the increase in pressure reduces the volume of the nitrocellulose membrane in each sampling pool, which in turn increases the number of labelling molecules per unit volume of the sampling pool, leading to higher conversion rates and higher sensitivity of the assay. At even higher pressure (e.g. 50N), the compression becomes too high and the majority of the sample liquid is forced away out of the sampling pools, leading to a higher resistance between the electrodes and impaired current flow.

Compression of the compressible portion 605 of the housing may in some embodiments be implemented through one or more corresponding features (releasing mechanism) in an electronic reader, such that upon insertion of the lateral flow test device 600 into the electronic reader, the compressible portion 605 is compressed by the one or more corresponding features of the electronic reader to displace the electrode array 609 from the initial position. Below describe various non-limiting examples of LFT device and electronic reader with corresponding features. It should be noted that, herein, a compressible portion simply refers to a section or portion of the housing that is to be compressed in order to displace the electrode array (or to displace the LFA strip if desired) from the initial position. While the compressible portion 605 is shown in FIG. 6 as being partially cut to allow compression, it is not considered essential, and uncut compressible portion for example formed of flexible material or completely unmodified compression portion are also possible.

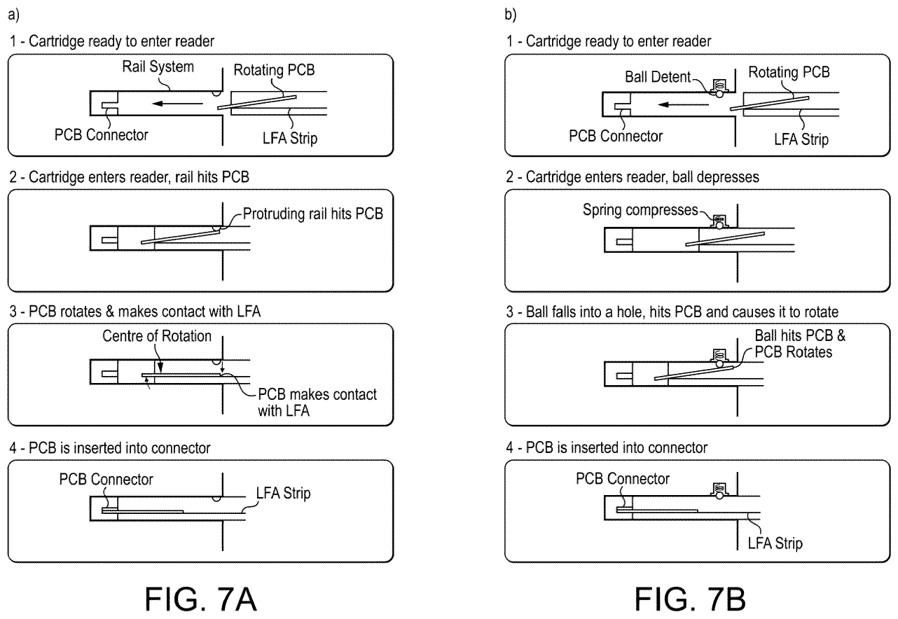

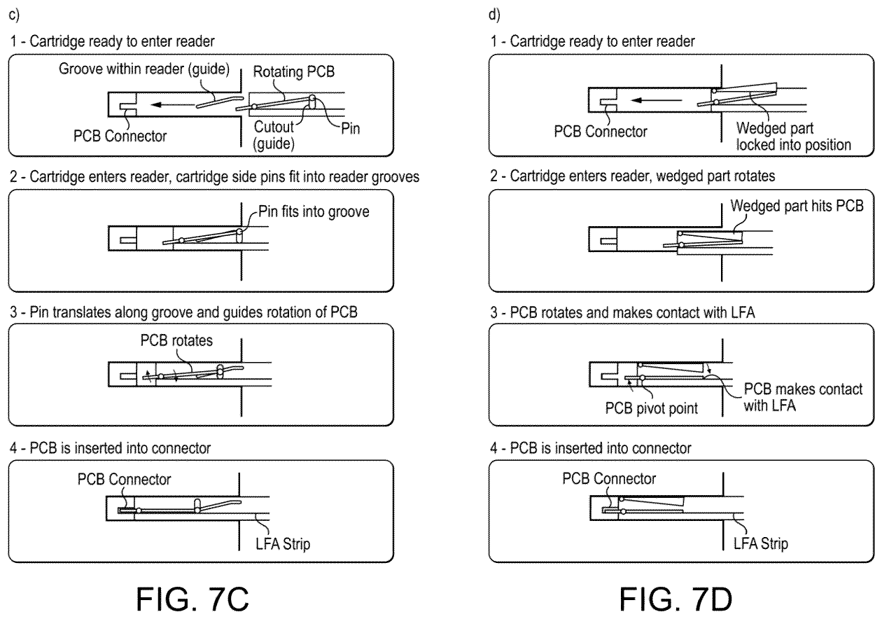

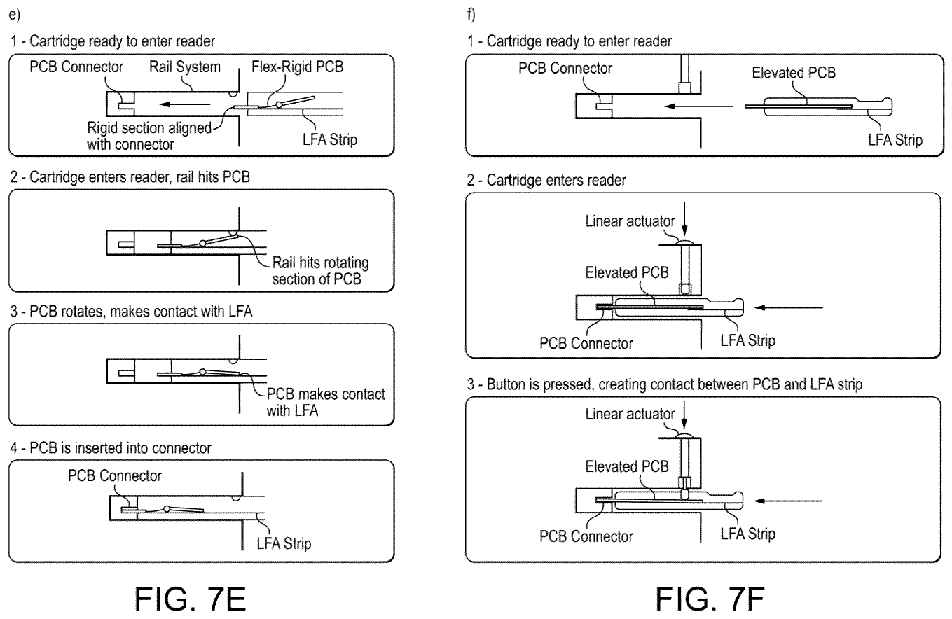

FIG. 7A shows an LFT device in which the electrode array is supported within the housing at an angle spaced apart from the LFA strip. A protruding rail provided to the top interior surface of the receiving portion of a corresponding electronic reader. As the LFT device is inserted into the electronic reader, the LFT device is moved into a position in which the protruding rail is above the compressible portion of the LFT device and the protruding rail engages the compressible portion to displace the electrode array, rotating the electrode array to enable contact between the electrode array and the LFA strip.

FIG. 7B shows an LFT device in which the electrode array is again supported within the housing at an angle spaced apart from the LFA strip. A spring compressed ball detent is provided to the receiving portion of a corresponding electronic reader, wherein the spring (or other resilient element) is provided within a recess with the ball bearing protruding into the interior of the receiving portion biased by the spring. As the LFT device is inserted into the electronic reader, the ball bearing is pushed into the recess, compressing the spring. Then, as the LFT device is inserted further, the compressible portion of the LFT device moves towards the recess and the ball bearing is released onto the compressible portion under the force of the compressed spring, compressing the compressible portion to displace the electrode array.

FIG. 7C shows an LFT device in which the electrode array is supported within the housing at an angle spaced apart from the LFA strip and the electrode array is held in place by a pin at one end which extends through a cut-out portion of the housing. A corresponding electronic reader is provided with a guide groove (or a pair of guide grooves if a pair of pins are provided one either side of the electrode array) that descends from the entrance of the receiving portion of the electronic reader towards a centre line of the receiving portion. As the LFT device is inserted into the electronic reader, the pin of the electrode array engages the guide groove, which guides the pin along the cut-out portion to rotate the electrode array and brings the electrode array into contact with the LFA strip.

FIG. 7D shows an LFT device in which the electrode array is supported within the housing at an angle spaced apart from the LFA strip. The housing of the LFT device is provided with a wedge-shaped portion pivoted at a front end of the LFT device and extends to the compressible portion of the housing. As the LFT device is inserted into an electronic reader, the receiving portion of the electronic reader pushes the wedge-shaped portion and eventually pushes the thick end of the wedge-shaped portion into the compressible portion of the housing to displace the electrode array, bringing the electrode array into contact with the LFA strip.

FIG. 7E shows an LFT device in which a flexible or partially flexible electrode array is used. In this example, the electrode array is supported at a division between the flexible section and the rigid section. A protruding rail is provided to an interior surface of the receiving portion of a corresponding electronic reader. As the LFT device is inserted, the housing of the LFT device glides over the protruding rail until the compressible portion reaches the protruding rail, at which point compression of the compressible portion pushes the rigid section of the electrode array, causing it to rotate towards the LFA strip and make contact. The flexible section of the electrode array allows a front rigid section to be positioned in alignment with a connector (communication port) of the electronic reader, while enabling a rear rigid section to be lifted away from the LFA strip.

FIG. 7F shows an LFT device in which the electrode array is supported in an elevated position spaced apart from the LFA strip. A rear section of the electrode array overlays a front section of the LFA strip at the position of the compressible portion of the housing. A corresponding electronic reader is provided with a linear actuator, such as a button, a lever or any other suitable and desirable mechanical activation mechanism, configured to extend inwards towards a centre line of the receiving portion of the electronic reader upon actuation. When the LFT device is inserted into the electronic reader, the linear actuator is actuated to press down on the compressible portion of the LFT device, thus displacing the electrode array to bring the electrode array into contact with the LFA strip. When in contact with the LFA strip, the electrode array may be put in a tilted position or, if the electrode array is flexible, the electrode array is allowed to flex over a distance of the LFT device.

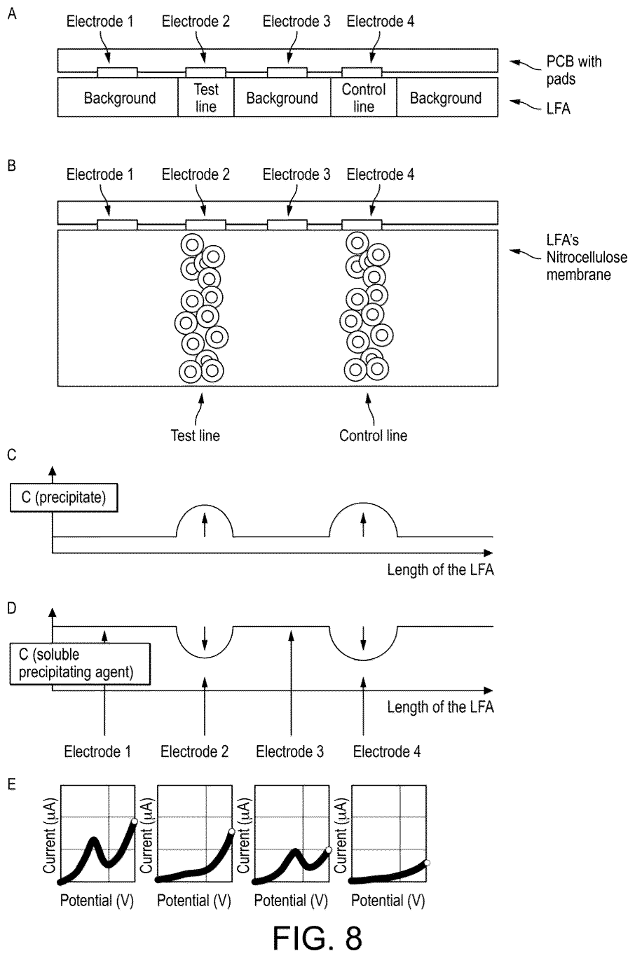

The signal readout is based on the working electrodes and counter electrodes being in contact with the nitrocellulose membrane, for example as shown in the embodiment of FIG. 8A in which the electrodes are on the underside of the PCB.

Where the present technology is implemented on an off the shelf LFT by a third-party manufacturer, the LFT is performed without any changes to existing LFT procedures. In some cases, if the sample type is such that it interferes with the chemistry of the LFT, this can be mitigated by the addition of a wash step where the LFT device is rinsed, e.g. with water (e.g. in the sink under tap water) before addition of the reagent. The wash step may be performed for as long as needed or desired, for example from a few seconds to a few minutes. The wash step removes unwanted species (e.g. Cl⁻) while introducing species that may be useful to silver enhancement reaction (e.g. water). In one embodiment, after following the LFT manufacturer's protocol, the nitrocellulose membrane may be washed with 50 μL of water or any other suitable buffer added via the sample port to remove excess electrolytes from the membrane and a signal enhancer such as a silver enhancer may be added. Typical silver enhancers available commercially require activation just before use, usually by combining a “developer” and an “enhancer” stock solutions in a 1:1 ratio. The developer enables spontaneous reduction of silver ions to metallic silver in the presence of gold nanoparticles and/or gold-plated electrodes. The resulting activated silver enhancer solution is then deposited on the membrane (50 μL) via the sample port and the reaction takes place within a 1-25 min timeframe. Contact between the electrodes and the nitrocellulose strip may be maintained throughout the LFA manufacturer's test, or the electrode array can be placed on top of the LFT strip after the LFT has been run and the silver has reacted with the AuNPs. This may be required in some situations to ensure the flow of the test is not disturbed. Contact between the membrane and the electrode array may be achieved by manual compression (squeezing the cassette to collapse internal struts) or by inserting the cassette into the reader to initialize the electrical readout.

Following the introduction of the activated silver solution, the AuNPs become coated with metallic silver, as illustrated in FIG. 8B. The distribution of precipitated metallic silver along the nitrocellulose membrane indicates the concentration of AuNPs due to the AuNPs catalysing the reduction of free silver ions, thus allowing the precipitation of metallic silver, as shown in FIG. 8C. The increased amount of silver precipitate leads to a corresponding decrease in free silver ions in the nitrocellulose membrane locally where AuNPs have formed immunocomplexes, as shown in FIG. 8D. Placing gold-plated PCB electrodes over nitrocellulose membrane initiates the reduction of silver ions on gold PCBs. The amount of available silver ions can be quantified by a potential sweep where the silver ions which have reduced on the gold electrodes are oxidized forming an oxidation peak observed in FIG. 8E. The height of the peak therefore correlates with the local concentration of the free silver ions in the nitrocellulose membrane. As accumulated AuNPs on the nitrocellulose membrane compete with the reduction of silver ions on the electrodes, the silver oxidation peak disappears over the test/control line in case of a positive sample, as shown in FIG. 8E.

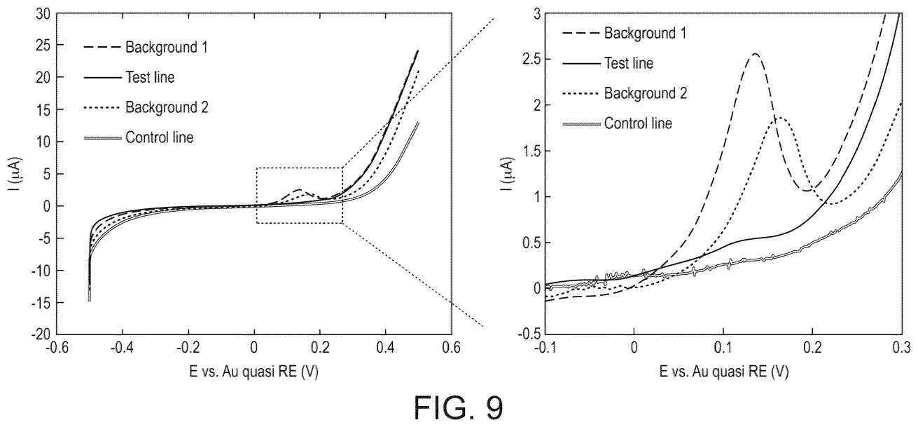

The electrochemical readout can be performed with any electrochemical technique such as voltammetric, amperometric, potentiometric or impedance-based methods. Linear sweep voltammetry (LSV) has been used successfully at 50 mV/s scan rate from −0.5 to +0.5 V against a reference potential such as vs. quasi-Au PCB RE, as shown in FIG. 9. The low starting potential of the LSV ensures the positive Ag+ ions in proximity of the electrode are first reduced on the electrode and deposited on the PCB surface, before the potential is increased and the oxidation peak can be observed between 0.1 and V vs. quasi-Au PCB RE. The potential continues to increase to +0.5 V vs. quasi-Au PCB RE ensuring complete oxidation of the silver on the PCB electrodes.

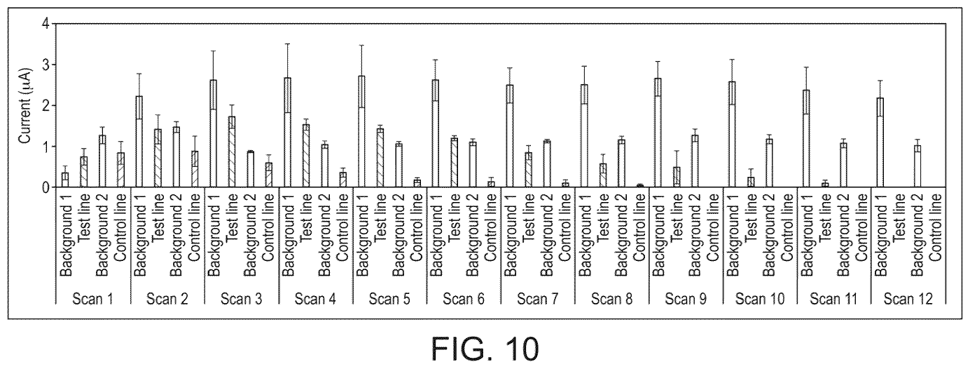

FIG. 10 shows the results of sequential scanning of four PCB electrodes where the first and the third electrodes overlay the background, the second overlays the test line, and the fourth overlays the control line, where both test line and control line are positive i.e., have accumulated AuPNs. The bars represent the average of three repeats, error bars are standard deviation. When the PCB electrodes are first brought into contact with the LFT membrane, the initial LSV scans do not exhibit the pattern presented in FIG. 9. In the first scan of all 4 electrodes, the peak heights do not follow any pattern, as can be seen in FIG. 10. Only when multiple LSV scans are performed the test line and control line current drop can be observed. Nevertheless, the silver oxidation peaks from electrodes overlayed with background remain stable throughout scans 2-12 and no drop in current can be observed.

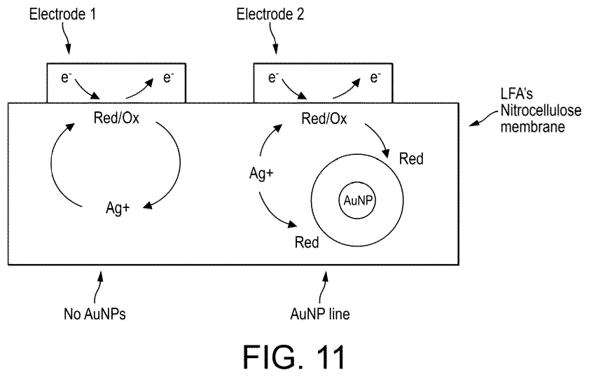

Due to repeating exposure to low and high potentials (through LSV measurement) silver ions are reduced/oxidized as well as attracted and repulsed away from the electrode. This increases the diffusion and promotes the moving ions to interact with AuNPs if these are present in the proximity of the electrode. The interaction of silver ions with AuNPs results in the reduction of silver on the AuNPs, competing with the reduction of silver ions on the PCB electrodes. As the AuNPs are not immobilized on the electrode surface but present in the bulk of the membrane, this is especially important to accurately evaluate the concentration of the silver ions in test/control lines. As schematically illustrated in FIG. 11, repeating LSV measurements produces potential cycling from low to high potentials causing repeating reduction and oxidation of silver as well as attraction and repulsion of silver ions. This promotes the diffusion of silver ions and allows for silver to react with AuNPs more efficiently.

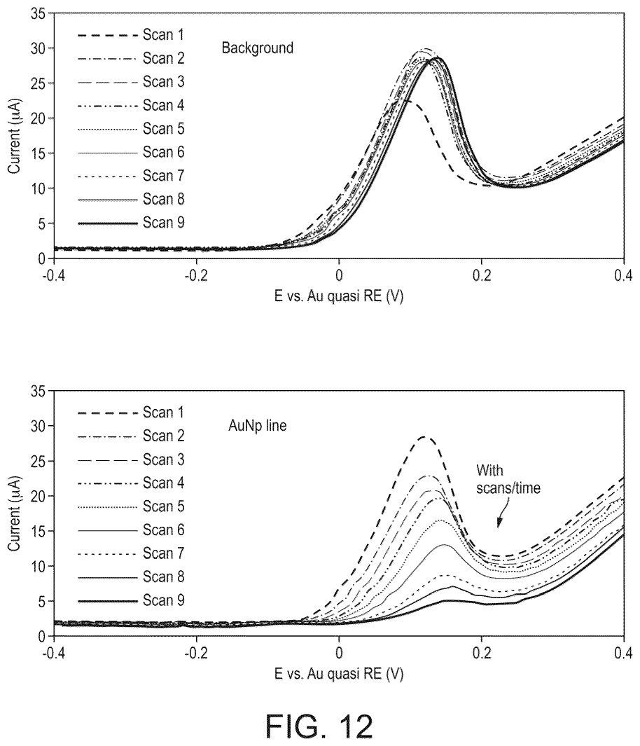

To increase the sensitivity of the assay, background current peak height is important since it is a deviation from these peak currents that demonstrates the presence of an electrochemical “signal”. In an effort to obtain a high signal-to-noise ratio, a deposition step can be used where a low potential such as −0.4 vs. quasi-Au PCB RE can be used for a predisposed time e.g. 10 sec to 1 min before the potential sweep. In this scenario, the positive silver ions are attracted to the negatively charged electrode and reduced on the electrode surface. Deposition potential and time are two parameters amongst others which can be regulated to obtain higher background currents. Furthermore, pulse based electrochemical techniques such as differential pulse voltammetry (DPV) can be used to detect low levels of silver ions. Electrochemical deposition and DVP enable a 10-fold increase in obtained current levels, which again decreases with the number of scans in a AuNP line, as shown in FIG. 12.

It should be noted that the methods described herein may include multiple independent potential sweeps as well as a coordinated combination of multiple potential sweeps.

If multiple potential sweeps are performed sequentially, the time difference between the electrode measurements may affect the catalytic event on the electrodes. For example, the high potential at the end of a potential sweep may oxidase one or more species, e.g. metallic silver, which precipitates on the surface of the catalytically active electrode. A possible mitigation is to perform a series of potential sweeps, where the initial cycle of potential sweeps is used to establish a baseline timepoint.

For example, a potential sweep may be performed on a first electrode, followed by the same potential sweep on a second electrode, a third electrode and a fourth electrode. This series of potential sweeps may be regarded as one cycle of potential sweeps. After completing each sweep of the first cycle, all silver species are oxidised in the proximity of the electrodes. After completing the first cycle of potential sweeps, further cycles may be repeated in which each electrode is incubated in the LFT device for the same controlled period of time.

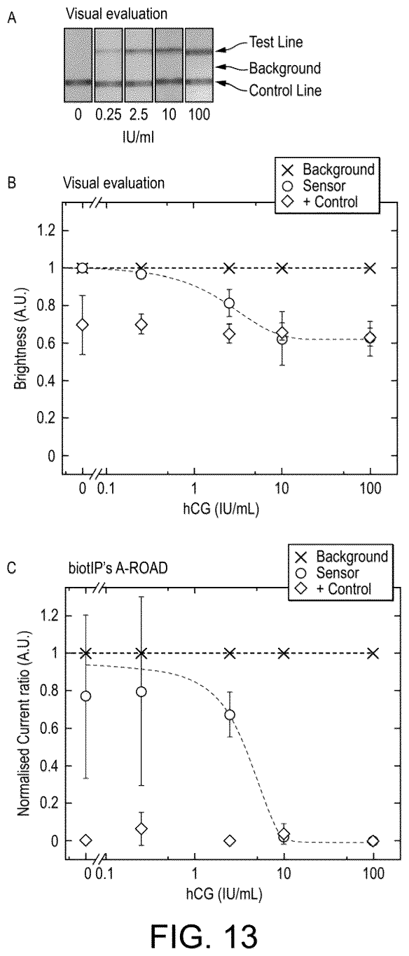

The method has been tested using commercially available hCG LFTs. FIGS. 10A, 10B and 10C show comparison of visual and electrochemical readout in a commercially available hCG LFT, where FIG. 13A represents visual tests with increasing hCG concentrations, FIG. 13B represents visual evaluation of brightness in test/control line in comparison to the background with brightness shown on the Y-axis being the ratio of test/control line divided by background, and FIG. 13C represents electrochemical evaluation on the same test strips of FIG. 13B with current ratio on the Y-axis being the ratio of test/control line current divided by the background. All datapoints have been performed in replicates (N=3), symbols represent the average, error bars showing standard deviation. In datapoints where error bars are not seen the symbol is larger than the error bars.

The tests have been performed and analysed visually using ImageJ software by reading the brightness of the test/control lines in comparison to background, shown in FIGS. 13A and 13B. Due to non-zero brightness of the background the signal drops to a minimum of 0.6 A.U. in positive hCG samples. When the same test was evaluated electrochemically, a signal drop to almost complete 0 is observed in samples with high hCG concentration, as shown in FIG. 13C. Similar sensing characteristics can be observed in both cases indicating the two methods can be used interchangeably, with electrochemical detection bypassing the subjective decision making of the LFT users.

Techniques described herein enable lateral flow tests to be conducted with improved accuracy and allow results to be objectively measured through the use of a signal enhancer that precipitates on the labelling particle that renders the test line measurable using an electrode array. As such, techniques described herein improves the effectiveness and usability of LFTs.

It will be clear to one skilled in the art that many improvements and modifications can be made to the foregoing exemplary embodiments without departing from the scope of the present techniques.

1. A lateral flow test device for performing a lateral flow test on a liquid sample, comprising:
a test strip which comprises:
a nitrocellulose membrane having a first capturing reagent disposed on a first surface along a test line, the first capturing reagent being configured to capture a first analyte in the liquid sample;
a sample pad disposed at a first end of the nitrocellulose membrane configured to receive the liquid sample;
a labelling reagent comprising a plurality of label molecules disposed on the first surface at a position between the sample pad and the test line, the label molecules being configured to bind to the first analyte;
an electrode array disposed over the nitrocellulose membrane configured to apply an electric potential across the first surface; and
a housing enclosing the test strip and the electrode array, the housing comprises a support element configured to support the electrode array and the test strip in an initial position wherein the electrode array is spaced apart from the test strip.

2. The device of claim 1, wherein the housing comprises a compressible portion configured to displace the electrode array from the initial position when the compressible portion is compressed.

3. The device of claim 2, wherein the support element comprises a pivot coupled to the electrode array to allow the electrode array to rotate.

4. The device of claim 3, wherein:
the support element further comprises a pin coupled to a rear end of the electrode array, the pin extending through a linear cut-out in the housing, wherein movement of the pin rotates the electrode array about the pivot; or
the housing further comprises a wedge element, wherein, upon compression, the wedge element compresses the compressible portion to rotate the electrode array about the pivot.

5-11. (canceled)

12. The device of claim 1, wherein the nitrocellulose membrane further comprises a second capture reagent disposed on the first surface along a control line, the second capture reagent being configured to capture the label molecules.

13. The device of claim 12, wherein the plurality of electrodes comprises one or more working electrodes arranged to overlay the test line and a background portion of the nitrocellulose membrane, the one or more working electrodes being configured to apply an electric potential on the first surface of the nitrocellulose membrane; and
wherein the plurality of electrodes comprises one or more counter electrodes each arranged to oppose a corresponding working electrode to complete an electric circuit; and
wherein the plurality of electrodes comprises one or more reference electrodes configured to act as a point of reference for the potential applied by the working electrodes; and
wherein the or each reference electrode is disposed adjacent a counter electrode.

14-21. (canceled)

22. The device of claim 1, further comprising a signal enhancer disposed on the first surface of the nitrocellulose membrane or the sample pad configured to precipitate on a surface of the label molecules for electrochemical detection of the label molecules.

23-30. (canceled)

31. The device of claim 1, further comprising:
an integrated electronic reader configured to generate an electrical signal through the electrode array to drive an electrochemical reaction in the test strip, and to receive a resulting electrochemical signal from the test strip through the electrode array; and
a power source configured to power the electrode array through the electronic reader.

32. The device of claim 31, further comprising a communication interface configured to communicate with an external electronic device to send the received electrochemical signal.

33. (canceled)

34. The device of claim 1, wherein the first capturing reagent is further disposed on the first surface the nitrocellulose membrane along a second test line at a concentration different from the first test line.

35. The device of claim 1, wherein the nitrocellulose membrane further comprises a third capturing reagent disposed on the first surface along a third test line, the third capturing reagent being configured to capture a third analyte in the liquid sample different from the first analyte.

36. The device of claim 1, wherein the electrode array comprises a plurality of pairs of corresponding electrodes, the plurality of corresponding electrodes being arranged such that a pair of corresponding electrodes of the plurality overlays a background of the nitrocellulose membrane and a pair of corresponding electrodes overlays the first test line, and optionally a pair of corresponding electrodes of the plurality overlays each of the control line and/or the second test line and/or the third test line.

37. An electronic reader for reading a result from a lateral flow test device, the lateral flow test device comprises a compressible portion, the electronic reader comprising:
a receiving portion for receiving the lateral flow test device comprising a releasing mechanism configured to compress the compressible portion of the lateral flow test device; and
a communication port configured to electrically couple with the lateral flow test device to generate an electrical signal for driving an electrochemical reaction within the lateral flow test device and to receive a resulting electrochemical signal indicative of the electrochemical reaction.

38-39. (canceled)

40. The electronic reader of claim 37, wherein the electronic reader further comprises an optical reader for reading a unique identifier on the lateral flow test device.

41. The electronic reader of claim 37, wherein the releasing mechanism comprises a linear actuator configured to compress the compressible portion of the lateral flow test device upon insertion of the lateral flow test device into the receiving portion.

42. The electronic reader of claim 37, wherein the electronic reader further comprises a timer configured to countdown a measurement time defined by a length of time required for measuring the result.

43-52. (canceled)

53. A method of performing a lateral flow test on a liquid sample using a lateral flow test strip, the lateral flow test strip comprising: a nitrocellulose membrane having a capturing reagent disposed on a first surface along a test line, the capturing reagent being configured to capture a predetermined analyte in the liquid sample; a sample pad disposed at one end of the nitrocellulose membrane configured to receive the liquid sample; and a labelling reagent comprising a plurality of label molecules disposed on the first surface between the sample pad and the test line, the label molecules being configured to bind to the predetermined analyte, the method comprising:
applying an electric potential across the first surface through an electrode array disposed over the nitrocellulose membrane to drive an electrochemical reaction in the lateral flow test strip; and
measuring a resulting electrochemical signal from the lateral flow test strip through the electrode array.

54-62. (canceled)

63. The method of claim 53, wherein measuring a resulting electrochemical signal from the lateral flow test strip comprises performing a potential sweep along the nitrocellulose membrane through the electrode array.

64. The method of claim 63, further comprising performing the potential sweep for at least two times, wherein a first cycle of potential sweep resets the electrochemical reaction and a second cycle of potential sweep measures the electrochemical signal.

65-67. (canceled)

68. The method of claim 53, wherein measuring a resulting electrochemical signal from the lateral flow test strip is performed using a linear sweep voltammetry method, optionally wherein the linear sweep voltammetry method comprises a potential sweep at a predetermined rate from a minimum potential to a maximum potential with respect to a reference potential, wherein optionally the predetermined rate is in a range of 10 mV per second to 1000 mV per second.

69-77. (canceled)
